(19) United States
(12) Patent Application Publication
(10) Pub. No.: US 2009/0203155 A1
(43) Pub. Date: Aug. 13, 2009

(54) LABELED PARTICLE OBTAINED BY IMMOBILIZING A FRAGMENTED ANTIBODY TO A LABELING SUBSTANCE

(76) Inventor: Hiroyuki CHIKU, Kanagawa (JP)

Correspondence Address: BIRCH STEWART KOLASCH & BIRCH, PO BOX 747, FALLS CHURCH, VA, 22040-0747, US

(21) Appl. No.: 12/369,568

(22) Filed: Feb. 11, 2009

(30) Foreign Application Priority Data
Feb. 12, 2008 (JP) 2008-029991

(51) Int. Cl. G01N 33/545; C07K 17/08

(52) U.S. Cl. 436/531; 530/391.5

(57) ABSTRACT

An object of the present invention is to provide a labeled particle having a high reactivity with an antigen and a suppressed non-specific adsorption, and an immunochromatographic method using the labeled particle. The present invention provides a labeled particle, wherein a fragmented antibody is immobilized to a labeling substance via a chemical bond.

### TECHNICAL FIELD

The present invention relates to a labeled particle having high reactivity with an antigen and exhibiting suppressed nonspecific adsorption and an immunochromatographic method using the labeled particle.

### BACKGROUND ART

Immunoassays are widely used as methods for qualitatively or quantitatively measuring the presence of an analyte existing in a biological sample such as urine or blood. Of these immunoassays, an immunochromatographic method is generally used with high frequency since its implementation is simple and enables short-time measurement.

The competitive reaction and the sandwich reaction are broadly used as immunoreactions to be employed in immunochromatographic methods. In particular, the sandwich reaction is mainly employed for an immunochromatographic method. In a typical example of the use of the sandwich reaction, the following procedures are performed to detect an analyte comprising an antigen in a sample. (1) A chromatographic medium having a reaction site is prepared by immobilizing a fine particle as a solid phase fine particle that has been sensitized with an antibody against an antigen that is an analyte on a chromatographic medium or by directly immobilizing the antibody on a chromatographic medium. (2) Meanwhile, a sensitization-target fine particle is prepared by sensitizing a labeled fine particle with an antibody capable of specifically binding to an analyte. (3) The sensitized and labeled fine particle is caused to migrate chromatographically on a chromatographic medium together with a sample.

The thus immobilized antibody is as an immobilized reagent at the reaction site formed on the chromatographic medium by the above procedures. The sensitized and labeled fine particle specifically binds to the reagent via an antigen that is an analyte. As a result, the presence, absence, or the amount of an analyte in a sample is measured by visually determining the presence, absence, or the degree of signals generated when the sensitized and labeled fine particle is captured at the reaction site.

In such immunochromatographic method, colloidal metal particles or colloidal metal oxide particles, colloidal nonmetal particles, and dye particles are used as fine particles for preparation of labeled fine particles.

When antibodies are bound to labeled particles, a method that is conventionally widely employed involves first mixing the antibodies with the labeled particles for physical adsorption and then blocking exposed portions of the labeled particles using a protein, a polymer, or the like. However, when physical adsorption is caused as described above, the orientation of the bound antibodies is varied, so that many antigen binding sites are oriented to the labeled particle side. Moreover, when adsorbed to the particles, some antibody structures may be altered. Antibodies in such a status cause decreased detection sensitivity or increased nonspecific adsorption.

In the case of some immunochromatographic methods, detection signals are amplified to avoid the problem of no antigens being detected because of low sensitivity (false negative). However, even in such case, a (false positive) problem can still arise since noise is enhanced due to signal amplification of nonspecifically adsorbed molecules, thus leading signals to be detected when no antigen is present.
Patent document 1: JP Patent Publication (Kokai) No. 7-146280 A (1995)
Patent document 2: JP Patent Publication (Kokai) No. 11-295313 A (1999)
Patent document 3: JP Patent Publication (Kohyo) No. 2005-512074 A

### DISCLOSURE OF THE INVENTION

To increase the detection sensitivity of an immunoassay using labeled particles under the circumstance with such problems, it is important to suppress the decrease of the antibody reactivity due to binding of antibodies to labeled particles, and to suppress the nonspecific adsorption of the labeled particles. An object of the present invention is to solve the above problems by realizing uniform orientation of antibodies to labeled particles, so as to provide a highly sensitive immunoassay.

As a result of intensive studies to achieve the above object, the present inventors have discovered that the reactivity can be improved and nonspecific adsorption can be suppressed by the use of labeled particles to which fragmented antibodies have been chemically bound. Thus, the present inventors have completed the present invention.

The present invention provides a labeled particle, wherein a fragmented antibody is immobilized to a labeling substance via a chemical bond.

Preferably, the fragmented antibody is an Fab fragment and/or an Fab′ fragment and/or an F(ab′)₂ fragment.

Preferably, the fragmented antibody is directly bound to the labeled particle, or is bound to the labeled particle via a hydrophilic polymer.

Preferably, the hydrophilic polymer contains an ethylene glycol group in at least a portion thereof.

Preferably, the polymer containing an ethylene glycol group in at least a portion thereof is at least one type selected from among polyethylene glycol and derivatives thereof.

Preferably, the fragmented antibody is bound to the labeled particle via an SH group of an antibody.

Preferably, the labeling substance is a metal colloid.

Preferably, the metal colloid is a gold colloid, a silver colloid, or a platinum colloid.

The present invention provides a sandwich immunochromatographic method which comprises developing a complex formed of an analyte and a labeled particle for the analyte on a porous carrier and capturing the analyte and the labeled particle at a reaction site on the porous carrier that has a second antibody against the analyte so as to detect the analyte, wherein the labeled particle is the labeled particle of the present invention as mentioned above.

Preferably, a labeling substance having an average particle size of 1 μm or more and 20 μm or less is detected.

Preferably, an analyte is detected via sensitization using a silver-containing compound and a reducing agent for silver ions.

Preferably, the reaction time for sensitization using the silver-containing compound and the reducing agent for silver ions is within 7 minutes.

Preferably, the number of the labeling substance at a detection site is 1×10⁶/mm³ or less.

Preferably, the labeling substance is a metal colloid.

According to the present invention, fragmented antibody-immobilized labeled particles having improved reactivity and exhibiting reduced nonspecific adsorption can be produced. Accordingly, increased detection sensitivity and decreased false positive results can be achieved, making it possible to obtain clear and precise assay results.

### BRIEF DESCRIPTION OF THE DRAWING

FIG. 1 is a plan view which schematically illustrates an embodiment of the immunochromatographic kit used in the present invention.

FIG. 2 is a longitudinal cross-sectional view which schematically illustrates a longitudinal cross-section of the immunochromatographic kit shown in FIG. 1.

FIG. 3 is a longitudinal cross-sectional view which schematically illustrates a longitudinal cross-section of another embodiment of the immunochromatographic kit used in the present invention.

1: Back adhesive sheet

2: Gold colloid antibody-retaining pad

3: Antibody-immobilized membrane

3a: Capturing site

31: Detection portion

32: Control portion

4: Absorbent pad

5: Sample-adding pad

6: Sensitization sheet

10: Immunochromatographic kit

### PREFERRED EMBODIMENTS OF THE INVENTION

The term “fragmented antibody” to be used in the present invention includes a Fab fragment and/or a Fab′ fragment and/or a F(ab′)₂ fragment.

An antibody comprises two heavy chains and two light chains and has a Y-shaped quadruplex structure as a basic structure. These heavy and light chains are linked via disulfide bonds so as to form heterodimers. Furthermore, the heterodimers are linked via two disulfide bonds, so as to form a Y-shaped heterotetramer. A V-shaped portion corresponding to the upper half of the Y shape is referred to as “Fab regions,” comprising two light chains and two heavy chains and binding to antigens with the tip portions of the two Fab regions. The Fab regions of heavy chains and Fc part are joined via a hinge region. The left and the right heavy chains are linked via disulfide bonds within the hinge region. The hinge region can be cleaved by a known method (enzyme treatment or chemical treatment). The thus generated antibody fragment is varied depending on the cleavage site of the hinge region. When the Fab regions contain the disulfide bonds of the hinge region, one F(ab′)₂ fragment in which two large Fabs are bound to each other, and a Fc fragment are generated. The F(ab′)₂ fragment contains the disulfide bond portion, so that it has a structure larger than that of two Fab fragments. Hence, the F(ab′)₂ fragment is referred to as Fab′ fragment for distinguishing from a Fab fragment. Furthermore, F(ab′)₂ fragment can also be converted into Fab′ fragment by treatment with a reducing agent such as 2-mercaptoethylamine. Also, when the Fab regions contain no disulfide bond in the hinge region, two Fab fragments and one Fc fragment are generated. Moreover, these antibody fragments can also be obtained using gene engineering techniques.

Fab fragments, F(ab′)₂ fragments, and Fab′ fragments obtained by such treatment contain antibody binding sites, however, unnecessary Fc fragments have been removed. Therefore, the use of these fragments in antigen detection results in decreased nonspecific adsorption and decreased noise. Thus, in the case of immunoassay such as ELISA, fragmented Fab fragments, F(ab′)₂ fragments, or Fab′ fragments tend to be used more often than complete antibody molecules.

In the case of conventional immunochromatographic methods, noise due to nonspecific adsorption is not a major problem because of low detection sensitivity. However, recently, sensitization is being performed by amplification of signals using an enzyme or the like, causing a problem such that noise is enhanced by signal amplification of a nonspecifically adsorbing molecule, resulting in false positives. In the present invention, preparation of an immunochromatographic kit using an antibody fragment makes it possible to suppress nonspecific adsorption to a degree greater than that in the case of preparation using a complete antibody molecule.

In the present invention, a fragmented antibody can be used regardless of animal species, subclasses, and the like. Examples of antibodies that can be used in the present invention include mouse IgG, mouse IgM, rat IgG, rat IgM, rabbit IgG, rabbit IgM, goat IgG, goat IgM, sheep IgG, and sheep IgM. They can be used as either polyclonal or monoclonal antibodies.

A method for specifically and chemically binding an antibody site to a labeled particle is not particularly limited. Examples of such method include a method that involves binding via an SH group of the hinge region of an antibody, a method that involves binding an antibody via a sugar chain of the antibody, and a method that involves binding an antibody via a functional group introduced in the antibody.

For example, a case is explained, in which an SH group of the hinge region of an antibody is used, through which the antibody is immobilized on a carrier via the SH group. H-chains are joined via an S—S bond in the hinge region of a mouse F(ab′)2 antibody IgG1, for example, and an SH group is generated upon reduction thereof. In the present invention, the thus generated SH group is used for immobilization. Therefore, the antibody in this case is fragmented to be Fab′ via reduction. In general reduction of an antibody, only the S—S bond of the hinge region is reduced to give an SH group, and S—S bonds at the other sites are not reduced. Therefore, only the SH group generated from the S—S bond of the hinge region is used for immobilization reaction, so that antibody immobilization is carried out via the specific site. Examples of a reducing agent to be used for reduction of an antibody are not particularly limited, and generally employed reducing agents can be used herein.

When an antibody is bound to a carrier via a sugar chain of the antibody, a sugar chain existing in the Fc portion of an antibody is bound to a carrier to which lectin or the like has been immobilized, for example, so that the antibody can be immobilized. This is because lectin is a sugar binding protein.

When an antibody is bound to a carrier via a functional group introduced into the antibody, a gene encoding six histidines is introduced into an antibody gene or a gene containing the antigen recognition portion of an antibody, and then the gene is expressed in Escherichia coli, yeast, an established cell line, or the like, for example. Meanwhile, when nickel is immobilized on the carrier surface using NTA (N-(5-amino-1-carboxypentyl) iminodiacetic acid) or the like and then an antibody expressing histidines on its end is added, the histidine portion is coordinated at the nickel. Thus, the antibody is specifically uniformly immobilized on the carrier surface (E. Hochuli, Journal of Chromatography, 1988, 444, 293 (1988)).

In the present invention, an antibody may be directly bound to a labeled particle or indirectly bound to the same via a linker. One end of such a linker has a binding group for binding to an antibody, or enables introduction of a binding group to the end. Examples of such binding group include, but are not particularly limited to, when binding is carried out via an SH group of the hinge region of an antibody, a maleimide group, a pyridyl disulfide group, a naphthyl disulfide group, active halogen, and thio phthalimide. Examples of a linker include a linear or branched alkyl group or piperazinyl group, linkers containing a hydrophilic group such as quaternary ammonium, and ethylene glycol-based compounds.

An example in which an antibody is bound to a labeled particle via a linker is as follows. First, since an SH group is exposed on the gold colloid surface, the colloid is mixed with a substance such as HS-PEGn-COOH. Thus, a gold colloid having PEG-COOH can be prepared. EDC and NHS are reacted with the thus prepared colloid, so that COOH group can be NHS-esterified. The NHS ester has high reactivity with an SH group. Hence, the NHS ester is mixed with an Fab′ antibody or the like in which SH groups exist, so that the Fab′ antibody can be bound to the gold colloid via the PEG chain.

Furthermore, a linker may be bound to a labeled particle via another substance. For example, a ligand and a receptor corresponding thereto are interposed between a labeled particle and a linker, and then an antibody may be bound to the labeled particle through them. For example, a ligand (or a receptor) is bound to one end of a linker, a receptor (or a ligand) corresponding to the ligand is bound to the surface of a labeled particle, and then they are bound, so that the antibody can be immobilized.

Examples of a ligand and a receptor include, but are not particularly limited to, avidin-biotin; hormones and their receptors such as insulin/insulin receptor, and a thyroid stimulating hormone (TSH)/TSH receptor; proteases and their inhibitors such as anhydrochymotrypsin/tryptophan as C-terminal amino acid, and subtilisin/a subtilisin inhibitor; proteases and their substrates such as anhydrotrypsin/a peptide containing arginine or lysine as C-terminal amino acid, peptides containing tyrosine and phenylalanine; and two types of DNA having complementary sequences.

Moreover, not only a ligand and a receptor, but also a macromolecular substance is interposed between a labeled particle and a linker and then antibody immobilization may be carried out. For example, a macromolecular substance is bound to a labeled particle in advance, and then a linker can be bound thereto. Examples of a macromolecular substance include proteins. Specifically, bovine serum albumin, casein, and the like can be used. In addition to these examples, examples of the same include sugar chains and synthetic polymers such as nylon.

In the present invention, a metal colloid label or a metallic sulfide label is used as a labeled particle, for example. Examples of the metal colloid label or the metallic sulfide label are not particularly limited and include, as a metal colloid label, a platinum coloid, a gold colloid, and a silver colloid; and as a metallic sulfide label, each sulfide of iron, silver, lead, copper, cadmium, bismuth, antimony, tin, and mercury. For example, a gold colloid and a silver colloid are preferred in that such a gold colloid with an appropriate particle diameter appears red and a silver colloid with an appropriate diameter appears yellow. The particle diameter of such a metal colloid preferably ranges from approximately 1 nm to 500 nm and further more preferably ranges from 5 nm to 100 nm, since a particularly strong color tone is obtained. When gold colloid particles are used as a metal colloid, commercially available gold colloid particles may be used. Alternatively, gold colloid particles can be prepared by a conventional method such as a method for reducing chloroauric acid with sodium citrate (e.g., Nature Phys. Sci., vol. 241, 20, (1973)). In addition to these examples, colored latex particles of organic polymers such as polystyrene and a styrene-butadiene copolymer, liposomes containing pigments, and microcapsules containing pigments, and the like can also be used as labeled particles. The average particle diameter of labeled particles (or a colloid) preferably ranges from 0.02 μm to 10 μm.

The fragmented antibody-immobilized labeled particles of the present invention are particularly preferably used as labeled particles for immunoassay. This is because a labeled particle having high reactivity and exhibiting suppressed nonspecific adsorption is required for use in measurement in the field of diagnosis that is required to be accomplished within a shorter time. A typical example of immunoassay is an immunochromatographic method. The present invention can also be used for an immunochromatographic method and an immunochromatographic kit and is composed as follows.

### 1. Immunochromatography

In general, immunochromatography is a method for determining and/or measuring an analyte, simply, rapidly and specifically, by the following means. That is to say, a chromatographic carrier having at least one reaction zone comprising an immobilizing reagent (an antibody, an antigen, etc.) capable of binding to an analyte is used as an immobilization phase. On this chromatographic carrier, a dispersed liquid formed by dispersion of a labeling substance used in detection, which is modified by a reagent capable of binding to an analytical target, is used as a mobile phase, and the mobile phase is moved in the chromatographic carrier in a chromatographic manner. At the same time, the aforementioned analytical target specifically binds to the labeling substance used in detection, and they reach the aforementioned reaction zone. At the aforementioned reaction zone, a complex of the aforementioned analytical target and the aforementioned labeling substance used in detection specifically binds to the aforementioned immobilizing reagent. Utilizing the phenomenon whereby the labeling substance used in detection is concentrated in the immobilizing reagent portion only when the analytical target exists in an analyzed solution, the presence of a product to be detected in the analyzed solution is qualitatively and quantitatively analyzed by visual observation or using an adequate apparatus.

The device for carrying out the immunochromatography in the present invention may comprise a compound containing silver and a reducing agent for silver ion. A signal is amplified by an amplification reaction using, as a core, a complex of the aforementioned analytical target and the aforementioned labeling substance used in detection binding to the aforementioned immobilizing reagent, so as to achieve high sensitivity. According to the present invention, a convenient, rapid and highly sensitive immunochromatography can be carried out without providing a metal ion and a reducing agent solution for amplification from the outside as in the case of a conventional immunochromatography.

### 2. Test Sample

The type of a test sample that can be analyzed by the immunochromatography of the present invention is not particularly limited, as long as it may comprise an analytical target. Examples of such a test sample include biological samples such as the body fluids of animals (particularly, a human) (e.g. blood, serum, plasma, spinal fluid, lacrimal fluid, sweat, urine, pus, runny nose, and sputum), excrements (e.g. feces), organs, tissues, mucous membranes, skin, a swab and a rinsed solution that are considered to contain them, and animals or plants themselves or the dried products thereof.

### 3. Pre-Treatment of Test Sample

In the immunochromatography of the present invention, the aforementioned test sample can directly be used. Otherwise, the aforementioned test sample can also be used in the form of an extract obtained by extracting it with a suitable extraction solvent, or in the form of a diluted solution obtained by diluting the aforementioned extract using a suitable diluent, or in the form of a concentrate obtained by concentrating the aforementioned extract by a suitable method. As the aforementioned extraction solvent, solvents used in common immunological analysis methods (e.g. water, a normal saline solution, a buffer, etc.) or water-miscible organic solvents that enable a direct antigen-antibody reaction as a result of dilution with the aforementioned solvents can be used.

### 4. Structure

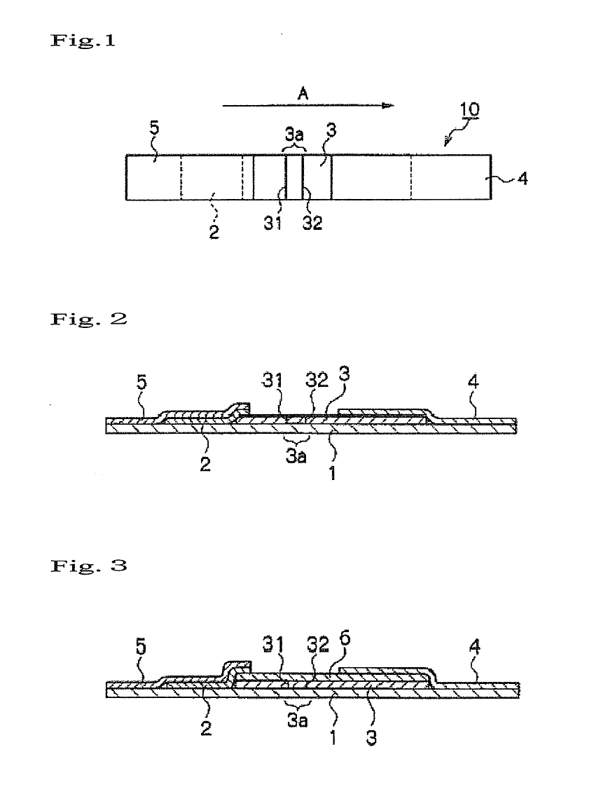

The type of an immunochromatographic strip that can be used in the immunochromatography of the present invention is not particularly limited, as long as it is an immunochromatographic strip that can be used in a common immunochromatography. For example, FIG. 1 schematically shows a plane view of the conventional immunochromatographic strip, for example. FIG. 2 is a longitudinal sectional view schematically showing a longitudinal section of the immunochromatographic kit as shown in FIG. 1. FIG. 3 schematically illustrates a longitudinal cross-section of another embodiment of the immunochromatographic strip which can be used in the present invention.

In an immunochromatographic strip 10 of the present invention, a sample-adding pad 5, a labeling substance-retaining pad (e.g. a gold colloid antibody-retaining pad) 2, a chromatographic carrier (e.g. an antibody-immobilized membrane) 3, and an absorbent pad 4 are disposed in this order on an adhesive sheet 5 from the upstream to the downstream of a development direction (a direction indicated with the arrow A in FIG. 1).

The chromatographic carrier 3 has a capturing site 3a and a detection zone (which is also referred to as a “detection portion”) 31 that is a region on which an antibody or an antigen specifically binding to an analytical target is immobilized. The chromatographic carrier 3 also has a control zone (which is also referred to as a “control portion”) 32 that is a region on which a control antibody or antigen is immobilized, as desired. Further, the detection zone 31 and the control zone 32 comprise organic silver salts used for amplification and reducing agents used for silver ion.

The labeling substance-retaining pad 2 can be produced by preparing a suspension containing a labeling substance, applying the suspension to a suitable absorbent pad (e.g. a glass fiber pad), and then drying it.

As the sample-adding pad 1, a glass fiber pad can be used, for example.

### 4-1. Label for Detection

In the method of the present invention, a labeled particle wherein a fragmented antibody is immobilized to a labeling substance via a chemical bond, is used as a label for detection.

### 4-2 Antibody

In the immunochromatography of the present invention, the type of an antibody having specificity for an analytical target is not particularly limited. Examples of an antibody used herein include fragments (for example, F(ab′)2, Fab, Fab′ or Fv) of an antiserum prepared from the serum of an animal immunized with the analytical target, an immunoglobulin fraction purified from the antiserum, and a monoclonal antibody obtained by cell fusion using the splenic cells of the animal immunized with the analytical target. Such an antibody may be prepared by a common method.

Representative methods for preparation of fragmented antibodies are the following two methods. First, when an antibody is treated with a papain enzyme, the antibody is denatured into two Fab fragments and one Fc fragment. Furthermore, when an antibody is treated with a pepsin enzyme, the antibody is denatured into F(ab′)₂ in which two Fab fragments are linked and an Fc fragment. Examples of an enzyme for preparation of a fragmented antibody include, other than the above enzymes, ficin, lysyl endopeptidase, V8 protease, bromelin, clostripain, metalloendopeptidase, and activated papain prepared by activation of papain. Furthermore, F(ab′)₂ can also be converted into Fab′ via treatment with a suitable reducing agent. The reducing agent foe use in the reduction of an antibody is not particularly limited, and any reducing agent which are usually used can be used. Examples thereof include mercaptoethanol, mercaptoethylamine, and dithiothreitol. Fab fragments, F(ab′)₂ fragments, and Fab′ fragments obtained by such treatment contain antibody binding sites, however, unnecessary Fc fragments have been removed.

### 4-3. Chromatographic Carrier

The chromatographic carrier is preferably a porous carrier. It is particularly preferably a nitrocellulose membrane, a cellulose membrane, an acetyl cellulose membrane, a polysulfone membrane, a polyether sulfone membrane, a nylon membrane, glass fibers, a nonwoven fabric, a cloth, threads or the like.

Usually, a substance used in detection is immobilized on a part of the chromatographic carrier to form a detection zone. The substance used in detection may be directly immobilized on a part of the chromatographic carrier via a physical or chemical bond. Alternatively, the substance used in detection may be bound physically or chemically to fine particles such as latex particles, and thereafter, the fine particles are immobilized on a part of the chromatographic carrier by trapping them thereon. After immobilization of the substance used in detection on the chromatographic carrier, the chromatographic carrier may preferably be subjected to a treatment for preventing unspecific adsorption, such as a treatment using an inert protein, and it may be then used.

### 4-4. Sample-Adding Pad

Examples of a material for the sample-adding pad include, but are not limited to, those having uniform characteristics, such as a cellulose filter paper, glass fibers, polyurethane, polyacetate, cellulose acetate, nylon, and a cotton cloth. A sample-adding portion not only acts to receive a sample containing the added analytical target, but also acts to filter off insoluble particles, etc. contained in the sample. Moreover, in order to prevent a decrease in analysis precision occurring during the analysis due to unspecific adsorption of the analytical target contained in the sample on the material of the sample-adding portion, the material constituting the sample-adding portion may be subjected to a treatment for preventing unspecific adsorption before use.

### 4-5. Labeling Substance-Retaining Pad

Examples of a material for the labeling substance-retaining pad include a cellulose filter paper, glass fibers, and a nonwoven fabric. Such a labeling substance-retaining pad is prepared by impregnating the pad with a predetermined amount of the labeling substance used in detection as prepared above and then drying it.

### 4-6. Absorbent Pad

The absorbent pad is a portion for physically absorbing the added sample as a result of the chromatographic migration and for absorbing and removing an unreacted labeling substance, etc. that is not immobilized on the detection portion of the chromatographic carrier. Examples of a material for the absorbent pad include water-absorbing materials such as a cellulose filter paper, a nonwoven fabric, a cloth or cellulose acetate. The chromatographic speed after the chromatographic leading end of the added sample has reached the absorbing portion varies depending on the material and size of the absorbent material, etc. Thus, a speed adequate for the measurement of the analytical target can be determined by selection of the material and size of the absorbent material.

### 5. Immunological Test Method

Hereinafter, a sandwich method which is specific embodiment of the immunochromatography of the present invention, will be described. In the sandwich method, an analytical target can be analyzed by the following procedures, for example, but the procedures are not particularly limited thereto. First, a primary antibody and a secondary antibody having specificity for an analytical target (an antigen) have previously been prepared by the aforementioned method. In addition, the primary antibody has previously been labeled. The second antibody is immobilized on a suitable insoluble thin-membrane support (e.g. a nitrocellulose membrane, a glass fiber membrane, a nylon membrane, a cellulose membrane, etc.), and it is then allowed to come into contact with a test sample (or an extract thereof) that is likely to contain the analytical target (the antigen). If the analytical target actually exists in the test sample, an antigen-antibody reaction occurs. This antigen-antibody reaction can be carried out in the same manner as that of an ordinary antigen-antibody reaction. At the same time of the antigen-antibody reaction or after completion of the reaction, an excessive amount of the labeled primary antibody is further allowed to come into contact with the resultant. If the analytical target exists in the test sample, an immune complex of the immobilized second antibody, the analytical target (antigen) and the labeled primary antibody is formed.

In the sandwich method, after completion of the reaction of the immobilized primary antibody, the analytical target (antigen) and the secondary antibody, the labeled secondary antibody that has not formed the aforementioned immune complex is removed. Subsequently, a region of the insoluble thin-membrane support, on which the second antibody has been immobilized, may be observed so as to detect or quantify the labeling substance, and detect the presence or absence of the analyte in the test sample or measure the amount of the analyte. Alternatively, a metal ion and a reducing agent are supplied, so that a signal from the labeling substance of the labeled primary antibody that has formed the aforementioned immune complex may be amplified and detected. Otherwise, a metal ion and a reducing agent are added to the labeled primary antibody, and they are simultaneously added to the thin-membrane support, so that a signal from the labeling substance of the labeled secondary antibody that has formed the aforementioned immune complex may be amplified.

### 6. Amplification Solution

An amplification solution that can be used in the present invention is what is called a developing solution as described in publications common in the field of photographic chemistry (e.g. “Kaitei Shashin kagaku no kiso, Ginen shashin hen (Revised Basic Photographic Engineering, silver salt photography),” (the Society of Photographic Science and Technology of Japan, Colona Publishing Co., Ltd.); “Shashin no kagaku (Photographic Chemistry),” (Akira Sasaki, Shashin Kogyo Shuppan); “Saishin Shoho Handbook (Latest Formulation Handbook),” (Shinichi Kikuchi et al., Amiko Shuppan); etc.).

In the present invention, any type of amplification solution can be used, as long as it is what is called a physical developing solution, which comprises silver ions, and such silver ions in the solution act as a core of development and reduction is carried out using a metal colloid as a center.

7. Compound that Contains Silver

The silver-containing compound used in the present invention may be an organic silver salt, an inorganic silver salt, or a silver complex.

The organic silver salt used in the present invention is an organic compound containing a reducible silver ion. Any one of an organic silver salt, an inorganic silver salt and a silver complex may be used as a compound containing a reducible silver ion in the present invention. For example, a silver nitrate, a silver acetate, a silver lactate, a silver butyrate, etc. have been known.

In addition, such a compound may be a silver salt or a coordination compound that forms a metallic silver relatively stable for light, when it is heated to 50° C. in the presence of a reducing agent.

The organic silver salt used in the present invention may be a compound selected from the silver salts of an azole compound and the silver salts of a mercapto compound. Such an azole compound is preferably a nitrogen-containing heterocyclic compound, and more preferably a triazole compound and a tetrazole compound. The mercapto compound is a compound having at least one mercapto group or thione group in the molecule thereof.

The silver salt of the nitrogen-containing heterocyclic compound of the present invention is preferably the silver salt of a compound having an imino group. Typical compounds include, but are not limited to, the silver salt of 1,2,4-triazole, the silver salt of benzotriazole or a derivative thereof (for example, a methylbenzotriazole silver salt and a 5-chlorobenzotriazole silver salt), a 1H-tetrazole compound such as phenylmercaptotetrazole described in U.S. Pat. No. 4,220,709, and imidazole or an imidazole derivative described in U.S. Patent No. 4,260,677. Among these types of silver salts, a benzotriazole derivative silver salt or a mixture of two or more silver salts is particularly preferable.

The silver salt of the nitrogen-containing heterocyclic compound used in the present invention is most preferably the silver salt of a benzotrialzole derivative.

The compound having a mercapto group or a thione group of the present invention is preferably a heterocyclic compound having 5 or 6 atoms. In this case, at least one atom in the ring is a nitrogen atom, and other atoms are carbon, oxygen, or sulfur atoms. Examples of such a heterocyclic compound include triazoles, oxazoles, thiazoles, thiazolines, imidazoles, diazoles, pyridines, and triazines. However, examples are not limited thereto.

Typical examples of the silver salt of the compound having a mercapto group or a thione group include, but are not limited to, the silver salt of 3-mercapto-4-phenyl-1,2,4-triazole, the silver salt of 2-mercapto-benzimidazole, the silver salt of 2-mercapto-5-aminothiazole, the silver salt of mercaptotriazine, the silver salt of 2-mercaptobenzoxazole, and the silver salt of compounds described in U.S. Pat. No. 4,123,274.

As such a compound having a mercapto group or a thione group of the present invention, a compound that does not contain a hetero ring may also be used. As such a mercapto or thione derivative that does not contain a hetero ring, an aliphatic or aromatic hydrocarbon compound having total 10 or more carbon atoms is preferable.

Among such mercapto or thione derivatives that do no contain a hetero ring, useful compounds include, but are not limited to, the silver salt of thioglycolic acid (for example, the silver salt of S-alkylthioglycolic acid having an alkyl group containing 12 to 22 carbon atoms) and the silver salt of dithiocarboxylic acid (for example, the silver salt of dithioacetic acid and the silver salt of thioamide).

An organic compound having the silver salt of carboxylic acid is also preferably used. It is straight-chain carboxylic acid, for example. Specifically, carboxylic acid containing 6 to 22 carbon atoms is preferably used. In addition, the silver salt of aromatic carboxylic acid is also preferable. Examples of such aromatic carboxylic acid and other carboxylic acids include, but are not limited to, substituted or unsubstituted silver benzoate (for example, silver 3,5-dihydroxybenzoate, silver o-methylbenzoate, silver m-methylbenzoate, silver p-methylbenzoate, silver 2,4-dichlorobenzoate, silver acetamide benzoate and silver p-phenylbenzoate), silver tannate, silver phthalate, silver terephthalate, silver salicylate, silver phenylacetate, and silver pyromellitate.

In the present invention, aliphatic acid silver containing a thioether group as described in U.S. Pat. No. 3,330,663 can also be preferably used. A soluble silver carboxylate having a hydrocarbon chain containing an ether bond or a thioether bond, or a soluble silver carboxylate having a sterically hindered substituent on an α-position (of the hydrocarbon group) or an ortho-position (of the aromatic group) can also be used. These silver carboxylates have an improved solubility in a coating solvent, which provides a coating material having little light scattering.

Such silver carboxylates are described in U.S. Pat. No. 5,491,059. All of the mixtures of the silver salts described therein can be used in the invention, as necessary.

The silver salt of sulfonate as described in U.S. Pat. No. 4,504,575 can also be used in the embodiment of the present invention.

Further, for example, the silver salt of acetylene described in U.S. Pat. No. 4,761,361 and No. 4,775,613 can also be used in the present invention. It can be provided as a core-shell type silver salt as described in U.S. Pat. No. 6,355,408. Such silver salt is composed of a core consisting of one or more silver salts and a shell consisting of one or more different silver salts.

In the present invention, another product useful as a non-photosensitive silver source is a silver dimer composite consisting of two different types of silver salts described in U.S. Pat. No. 6,472,131. Such a non-photosensitive silver dimer composite consists of two different types of silver salts. When the aforementioned two types of silver salts include a linear saturated hydrocarbon group as a silver ligand, a difference in the numbers of carbon atoms of the ligands is 6 or greater.

The organic silver salt is contained as silver generally in an amount of 0.001 to 0.2 mol/m², and preferably 0.01 to 0.05 mol/m², in terms of the silver amount.

The inorganic silver salt or the silver complex used in the present invention is a compound containing a reducible silver ion. Preferably, such an inorganic silver salt or a silver complex is an inorganic silver salt or a silver complex, which forms metallic silver relatively stable for light, when the salt or complex is heated to 50° C. or higher in the presence of a reducing agent.

Examples of the inorganic silver salt used in the present invention include: a silver halide (such as silver chloride, silver bromide, silver chlorobromide, silver iodide, silver chloroiodide, silver chloroiodobromide, and silver iodobromide); the silver salt of a silver thiosulfate (e.g. a sodium salt, a potassium salt, an ammonium salt, etc.); the silver salt of a silver thiocyanate (e.g. a sodium salt, a potassium salt, an ammonium salt, etc.); and the silver salt of a silver sulfite (e.g. a sodium salt, a potassium salt, an ammonium salt, etc.).

The inorganic silver salt used in the present invention is preferably a silver halide or silver nitrate.

A method for forming the particles of the silver halide used in the invention is well known in the photographic industry. For example, methods described in Research Disclosure No. 17029, June 1978, and U.S. Pat. No. 3,700,458 may be used. Specifically, such a silver halide may be prepared by adding a silver-supplying compound (for example, a silver nitrate) and a halogen-supplying compound to a solution of a gelatin or other polymers.

The particle size of the silver halide is preferably very small in order to reduce examination noise. Specifically, the size is preferably 0.20 μm or less, more preferably 0.10 μm or less, and even more preferably in the range of nanoparticles. The term “particle size” is used herein to mean a diameter of a circular image having the same area as the projected area of the silver halide particle (the projected area of the main plane in the case of a tabular particle).

A silver thiosulfate, a silver thiocyanate, and a silver sulfite can also be prepared in the same manner as the formation of silver halide particles, by mixing a silver-supplying compound (such as a silver nitrate) with a thiosulfate (e.g. a sodium salt, a potassium salt, an ammonium salt, etc.), a thiocyanate (e.g. a sodium salt, a potassium salt, an ammonium salt, etc.), and a sulfite (e.g. a sodium salt, a potassium salt, an ammonium salt, etc.), respectively.

In general, if the concentration of silver ion in the amplification solution is too high, such silver ion is reduced in the amplification solution. In order to prevent such a phenomenon, a complexing agent may be used to cause the silver ion to form a complex. As such a complexing agent, amino acids such as glycine and histidine, heterocyclic bases, imidazole, benzimidazole, pyrazole, purine, pyridine, aminopyridine, nicotinamide, quinoline, and other similar aromatic heterocyclic compounds have been known. These compounds are described in E.P. Patent No. 0293947, for example. Further, as a complex salt-forming agent, thiosulfate, thiocyanate, and the like can also be used. Specific examples of the silver complex used in the present invention include a complex of a thiosulfate and a silver ion, a complex of a thiocyanate and a silver ion, a composite silver complex thereof, a complex of a sugar thione derivative and a silver ion, a complex of a cyclic imide compound (e.g. uracil, urazole, 5-methyluracil, barbituric acid, etc.) and a silver ion, and a complex of a 1,1-bissulfonylalkane and a silver ion. A preferred silver complex used in the invention is a complex of a cyclic imide compound (e.g. uracil, urazole, 5-methyluracil, barbituric acid, etc.) and a silver ion.

The silver complex used in the present invention may be prepared by a generally-known salt forming reaction. For example, the silver complex may be prepared by mixing in water or a water-miscible solvent a water-soluble silver supplier (such as a silver nitrate) with a ligand compound corresponding to the silver complex. The prepared silver complex can be used, after salts generated as by-products have been removed by a known desalting method such as dialysis or ultrafiltration.

The inorganic silver salt or the silver complex is contained as silver generally in an amount of 0.001 to 0.2 mol/m², and preferably 0.01 to 0.05 mol/m², in terms of the silver amount.

When an inorganic silver salt or a silver complex is used, a solvent for them is preferably used. The solvent used in the present invention is preferably a compound used as a ligand for forming a silver complex described in the above paragraphs for the “silver complex.” Examples of such a compound used as a solvent in the present invention include a thiosulfate, a thiocyanate, a sugar thione derivative, a cyclic imide compound, and a 1,1-bissulfonylalkane. The solvent used in the present invention is more preferably a cyclic imide compound such as uracil, urazole, 5-methyluracil, or barbituric acid. The solvent used in the present invention is preferably used at a molar ratio of 0.1 to 10 moles with respect to silver ions.

### 8. Reducing Agent Used for Silver Ion

As a reducing agent used for silver ion, either inorganic or organic materials capable of reducing silver(I) ion to silver, or the mixtures thereof, may be used.

As an inorganic reducing agent, reducible metal salts and reducible metal complex salts whose valence can be changed with metal ions such as Fe²⁺, V²⁺ or Ti³⁺ have been known. These salts can be used in the present invention. When such an inorganic reducing agent is used, it is necessary to form a complex with the oxidized ion or reduce it, so as to remove or detoxify the oxidized ion. For example, in a system using Fe⁺² as a reducing agent, citric acid or EDTA is used to form a complex with Fe³⁺ as an oxide, so as to detoxify it.

In the present system, such an inorganic reducing agent is preferably used. The metal salt of Fe²⁺ is more preferable.

Developing agents used for wet-process silver halide photographic-sensitized materials (for example, methyl gallate, hydroquinone, substituted hydroquinone, 3-pyrazolidones, p-aminophenols, p-phenylenediamines, hindered phenols, amidoximes, azines, catechols, pyrogallols, ascorbic acid (or derivatives thereof), and leuco dyes), or other materials known to those skilled in the art (see, for example, U.S. Pat. No. 6,020,117 (Bauer et al.)) may be used in the present invention.

The term “ascorbic acid reducing agent” means a complex of ascorbic acid and a derivative thereof. Ascorbic acid reducing agents are described in many publications, as described below, including, for example, U.S. Pat. No. 5,236,816 (Purol et al.) and publications cited therein.

The reducing agent used in the present invention is preferably an ascorbic acid reducing agent. Useful ascorbic acid reducing agents include ascorbic acid, an analogue thereof, an isomer thereof, and a derivative thereof. Examples of such compounds include the following compounds. However, examples are not limited thereto.

Examples of such compounds include D- or L-ascorbic acid and a sugar derivative thereof (for example, γ-lactoascorbic acid, glucoascorbic acid, fucoascorbic acid, glucoheptoascorbic acid, and maltoascorbic acid), sodium ascorbate, potassium ascorbate, isoascorbic acid (or L-erythroascorbic acid), and a salt thereof (for example, an alkali metal salt, an ammonium salt, or salts known in the art), and endiol-type ascorbic acid, enaminol-type ascorbic acid and thioenol-type ascorbic acid such as compounds described in U.S. Pat. No. 5,498,511, EP-A-0585,792, EP-A 0573700, EP-A 0588408, U.S. Pat. Nos. 5,089,819, 5,278,035, 5,384,232 and 5,376,510, JP 7-56286, U.S. Pat. No. 2,688,549, and Research Disclosure 37152 (March, 1995).

Among these compounds, D-, L-, and D,L-ascorbic acid (and an alkali metal salt thereof), and isoascorbic acid (and an alkali metal salt thereof) are preferable. Moreover, a sodium salt is a preferred salt thereof. If necessary, a mixture of these reducing agents may also be used.

A hindered phenol may be preferably used singly or in combination with one or more gradation-hardening reducing agents and/or contrast enhancers.

A hindered phenol is a compound having only one hydroxyl group on a benzene ring and also having at least one substituent at the ortho-position relative to the hydroxyl group. The hindered phenol reducing agent may have plural hydroxyl groups, as long as the hydroxyl groups are located on different benzene rings.

Examples of the hindered phenol reducing agent include binaphthols (that is, dihydroxybinaphthols), biphenols (that is, dihydroxybiphenols), bis(hydroxynaphthyl)methanes, bis(hydroxyphenyl)methanes (that is, bisphenols), hindered phenols, and hindered naphthols, each of which may be substituted.

Typical binaphthols include, but are not limited to, 1,1′-bi-2-naphthol, 1,1′-bi-4-methyl-2-naphthol, and compounds described in U.S. Pat. Nos. 3,094,417 and 5,262,295.

Typical biphenols include, but are not limited to, 2-(2-hydroxy-3-t-butyl-5-methylphenyl)-4-methyl-6-n-hexylphenol, 4,4′-dihydroxy-3,3′,5,5′-tetra-t-butylbiphenyl, 4,4′-dihydroxy-3,3′,5,5′-tetramethylbiphenyl, and compounds described in U.S. Pat. No. 5,262,295.

Typical bis(hydroxynaphthyl)methanes include, but are not limited to, 4,4′-methylenebis(2-methyl-1-naphthol) and compounds described in U.S. Pat. No. 5,262,295.

Typical bis(hydroxyphenyl)methanes include, but are not limited to, bis(2-hydroxy-3-t-butyl-5-methylphenyl)methane (CAO-5), 1,1′-bis(2-hydroxy-3,5-dimethylphenyl)-3,5,5-trimethyl hexane (NONOX or PERMANAX WSO), 1,1′-bis(3,5-di-t-butyl-4-hydroxyphenyl)methane, 2,2′-bis(4-hydroxy-3-methylphenyl) propane, 4,4′-ethylidene-bis(2-t-butyl-6-methylphenol), 2,2′-isobutylidene-bis(4,6-dimethylphenol) (LOWINOX 221B46), 2,2′-bis(3,5-dimethyl-4-hydroxyphenyl)propane, and compounds described in U.S. Pat. No. 5,262,295.

Typical hindered phenols include, but are not limited to, 2,6-di-t-butylphenol, 2,6-di-t-butyl-4-methylphenol, 2,4-di-t-butylphenol, 2,6-dichlorophenol, 2,6-dimethylphenol, and 2-t-butyl-6-methylphenol.

Typical hindered naphthols include, but are not limited to, 1-naphthol, 4-methyl-1-naphthol, 4-methoxy-1-naphthol, 4-chloro-1-naphthol, 2-methyl-1-naphthol, and compounds described in U.S. Pat. No. 5,262,295.

Moreover, other compounds disclosed as reducing agents include amidoximes (for example, phenylamidoxime), 2-thienylamidoxime, p-phenoxyphenylamidoxime, a combination of an aliphatic carboxylic allyl hydrazide and ascorbic acid (for example, a combination of 2,2′-bis(hydroxymethyl)-propionyl-β-phenyl hydrazide and ascorbic acid), a combination of a polyhydroxybenzene and at least one of hydroxylamine, reductone and hydrazine (for example, a combination of hydroquinone and bis(ethoxyethyl)hydroxylamine), piperidi-4-methylphenylhydrazine, hydroxamic acids (for example, phenylhydroxamic acid, p-hydroxyphenylhydroxamic acid, and o-alaninehydroxamic acid), a combination of an azine and a sulfonamidophenol (for example, a combination of phenothiazine and 2,6-dichloro-4-benzenesulfonamidophenol), α-cyanophenylacetic acid derivatives (for example, ethyl-α-cyano-2-methylphenylacetic acid and ethyl-α-cyanophenylacetic acid), bis-o-naphthol (for example, 2,2′-dihydroxy-1-binaphthyl, 6,6′-dibromo-2,2′-dihydroxy-1,1′-binaphthyl, and bis(2-hydroxy-1-naphthyl)methane), a combination of bis-naphthol and a 1,3-dihydroxybenzene derivative (for example, 2,4-dihydroxybenzophenone and 2,4-dihydroxyacetophenone), 5-pyrazolones (for example, 3-methyl-1-phenyl-5-pyrazolone), reductones (for example, dimethylaminohexose reductone, anhydrodihydro-aminohexose reductone, and anhydrodihydro-piperidone-hexose reductone), indane-1,3-diones (for example, 2-phenylindane-1,3-dione), chromans (for example, 2,2-dimethyl-7-t-butyl-6-hydroxychroman), 1,4-dihydroxypyridines (for example, 2,6-dimethoxy-3,5-dicarbetoxy-1,4-dihydropyridine), ascorbic acid derivatives (1-ascorbic palmitate, ascorbic stearate), unsaturated aldehydes (ketones), and 3-pyrazolidones.

Examples of a reducing agent that can be used in the present invention include substituted hydrazines such as sulfonyl hydrazines described in U.S. Pat. No. 5,464,738. Other useful reducing agents are described, for example, in U.S. Pat. Nos. 3,074,809, 3,094,417, 3,080,254 and 3,887,417. Auxiliary reducing agents described in U.S. Pat. No. 5,981,151 are also useful.

The reducing agent may be a combination of a hindered phenol reducing agent and a compound selected from various auxiliary reducing agents such as those mentioned below. In addition, a mixture of such a combined agent plus a contrast enhancer (that is, a mixture of the 3 components) is also useful. As such an auxiliary reducing agent, it is possible to use trityl hydrazide and formyl-phenyl hydrazide described in U.S. Pat. No. 5,496,695.

A contrast enhancer may be used in combination with the reducing agent. Useful contrast enhancers include, but are not limited to, hydroxylamines (including hydroxylamine and alkyl- and aryl-substituted derivatives thereof), alkanolamines and phthalic ammonium described in U.S. Pat. No. 5,545,505, hydroxamic acid compounds described in U.S. Pat. No. 5,545,507, N-acylhydrazine compounds described in U.S. Pat. No. 5,558,983, and hydrogen atom donor compounds described in U.S. Pat. No. 5,637,449.

Not all combinations of reducing agents and organic silver salts are equally effective. A preferred combination is a benzotriazole silver salt used as an organic silver salt, a substituted compound thereof or a mixture thereof, with an ascorbic acid reducing agent used as a reducing agent.

The reducing agent of the present invention may be contained in an amount of 1 mass % to 10 mass % (dry mass) based on the amount of silver in organic silver. When the reducing agent is added to a layer other than the layer containing the organic silver salt in a multilayer structure, the amount of the reducing agent is slightly higher, and it is desirably from approximately 2 mass % to approximately 15 mass %. An auxiliary reducing agent is contained in an amount of about 0.001 mass % to 1.5 mass % (dry weight).

### 9. Other Auxiliary Agents

Other auxiliary agents contained in the amplification solution may include a buffer, an antiseptic such as an antioxidant or an organic stabilizer, and a speed regulator. Examples of a buffer used herein include buffers comprising acetic acid, citric acid, sodium hydroxide, a salt thereof, or tris(hydroxymethyl)aminomethane, and other buffers used in ordinary chemical experiments. Using these buffers as appropriate, the pH of the amplification solution can be adjusted to the optimal pH.

The present invention will be more specifically described in the following examples. However, these examples are not intended to limit the scope of the present invention.

### EXAMPLES

Each immunochromatographic kit was prepared by the following method.

### (1) Preparation of Fab′ Anti-Influenza Antibody

### 1. Preparation of F(ab′)₂ Anti-Influenza A Virus Antibody

An anti-influenza A virus antibody (Product No. 7307, Medix Biochemica) was used, and F(ab′)₂ anti-influenza A virus antibody was prepared using an ImmunoPure® IgG1 Fab and F(ab′)₂ Preparation Kit (Product No. 44880, Pierce).

### 2. Preparation of F(ab′)₂ Anti-Influenza B Virus Antibody

An anti-influenza B virus antibody (Product No. 1131 (ViroStat, Inc.)) and lysyl endopeptidase (Product No. 125-05061, Wako Pure Chemical Industries, Ltd.) were diluted with a 50 mM Tris-Hcl buffer (pH 8.5) to a molar ratio of 1: 100, followed by 3 hours of reaction at 37° C. Subsequently, the F(ab′)₂ antibody was purified using an ImmunoPure (A) IgG Purification Kit (Product No. 44667, Pierce).

### 3. Preparation of Fab′ Anti-Influenza Virus Antibody

Both F(ab′)₂ anti-influenza A virus and B virus antibodies were separately subjected to dialysis overnight at 4° C. with a 0.1 M sodium phosphate buffer (pH 6.0) and 5 mM EDTA. The thus dialyzed antibodies were each adjusted at 0.5 mg/mL, mercaptoethylamine was added to each solution to a final concentration of 6 mg/mL, followed by 1 hour of reaction at room temperature. Thus, Fab′ antibodies were prepared. The reaction products were subjected to buffer exchange with PBS buffer using AmiconUltra-4 (MWCO 30,000), so that mercaptoethylamine that had not undergone a reaction was removed.

### (2) Preparation of Antibody-Labeled Gold Colloid

### Comparative Example 1 Preparation of Gold Colloid to which Fab′ Antibody was Physically Adsorbed

1. Preparation of Fab′ Antibody in which SH Group was Blocked

For physical adsorption of Fab′ antibodies to a gold colloid (gold particles) as a comparative example, SH groups were blocked by the following procedure. 1 mL of 1 mg/mL N-ethylmaleimide (Product No. 23030, Pierce) was added to 1 mL of the thus prepared Fab′ antibody solution (the concentration was adjusted to 1 mg/mL), followed by 2 hours of reaction at room temperature, so that SH groups were blocked. The reaction product was subjected to buffer exchange with PBS buffer using AmiconUltra-4 (MWCO 30,000), so that N-ethylmaleimide that had that had not undergone a reaction was removed.

2. Preparation of Gold Colloid to which Fab′ Antibody was Physically Adsorbed

The following similar procedures were carried out separately for the A antibody and the B antibody.

1 mL of 300 μg/mL Fab′ antibody solution (in which SH had been blocked) was added to a gold colloidal solution having pH adjusted by addition of 1 mL of a 50 mM Borax buffer (pH 8.5) to 9 mL of a 50-nm diameter gold colloidal solution (EM.GC50, BBI), followed by stirring. The solution was allowed to stand for 10 minutes and then 550 μL of 1% polyethylene glycol (PEG Mw. 20000, Product No. 168-11285, Wako Pure Chemical Industries, Ltd.) aqueous solution was added to the solution, followed by stirring. Subsequently, 1.1 mL of a 10% bovine serum albumin (BSA FractionV, Product No. A-7906, SIGMA) aqueous solution was added to the resultant, followed by stirring. The solution was centrifuged at 8000×g and 4° C. for 30 minutes (himacCF16RX, Hitachi). The supematant was removed so that approximately 1 mL of the solution remained. The gold colloid was dispersed again using an ultrasonic washing machine. Subsequently, the resultant was then dispersed in 20 mL of a gold colloidal stock solution (20 mM Tris-HCl buffer (pH 8.2), 0.05% PEG (Mw.20000), 150 mM NaCl, 1 % BSA, and 0.1 % NaN₃) and then centrifuged again at 8000×g and 4° C. for 30 minutes. The supernatant was removed so that approximately 1 mL of the solution remained. The gold colloid was dispersed again using an ultrasonic washing machine, so that an antibody-labeled gold colloidal (50 nm) solution was obtained.

### Example 1

### Preparation of Gold Colloid to which Fab′ Antibody was Directly Immobilized Via SH Group

The thus prepared Fab′ antibody was adjusted to 0.5 mg/mL. 1 mL of the solution was mixed with a 50-nm diameter gold colloidal solution and then a reaction was carried out for 1 hour at room temperature for immobilization. A 1 % polyethylene glycol (PEG Mw.20000) aqueous solution (500 μL) was added to the reaction solution and then the solution was stirred. Subsequently, 1.0 mL of a 10 % bovine serum albumin aqueous solution was added and then the solution was stirred. The solution was centrifuged at 8000×g and 4° C. for 30 minutes. The supernatant was removed so that approximately 1 mL of the solution remained. The gold colloid was dispersed again using an ultrasonic washing machine. Subsequently, the resultant was then dispersed in 20 mL of a gold colloidal stock solution (20 mM Tris-HCl buffer (pH 8.2), 0.05% PEG (Mw. 20000), 150 mM NaCl, 1% BSA, and 0.1% NaN₃) and then centrifuged again at 8000×g and 4° C. for 30 minutes. The supernatant was removed so that approximately 1 mL of the solution remained. The gold colloid was dispersed again using an ultrasonic washing machine, so that an antibody-labeled gold colloidal solution was obtained.

By confirming that no antibody was eluted when the gold colloid was mixed with a 0.1% SDS solution, it was confirmed that the antibody could be bound via the SH group to the thus prepared labeled gold colloid.

### Example 2

### Preparation of Gold Colloid to which Fab′ Antibody was Immobilized Via SH Group Using PEG Polymer

9 mL of a 50-nm-diameter gold colloidal solution was mixed with 1 mL of 1 mM Thiol-dPEG₄ acid (Product No. QB10247a, Quanta), and then a reaction was carried out at room temperature for 18 hours, in order to treat the surface with PEG. 500 μL of EDC (0.2M) (Product No. E1769, Sigma-Aldrich Corporation) and 500 μL of 0.05 M NHS (Product No. 130672, Aldrich) were added to the reaction solution, and then reaction was carried out at room temperature for 3 hours, so that COOH groups were NHS-esterified. The solution was centrifuged at 8000×g and 25° C. for 15 minutes. The supernatant was removed so that approximately 1 mL of the solution remained and then the gold colloid was dispersed again using an ultrasonic washing machine. Subsequently, the resultant was dispersed in 20 mL of 50 mM KH₂PO₄ buffer (pH 7.0) and then centrifuged again at 8000×g and 25° C. for 15 minutes. The supernatant was removed so that approximately 1 mL of the solution remained and then the gold colloid was dispersed again using an ultrasonic washing machine. The resultant was adjusted using 50 mM KH₂PO₄ buffer (pH 7.0) to a total of 9 mL.

1 mL of the Fab′ antibody prepared in 1 was added to the gold colloidal solution, followed by reaction at room temperature for 2 hours. Thereafter, 1 mM amino-dPEG₄ alcohol (Product No. QB10240a, Quanta) was added and then reaction was carried out at room temperature for 1 hour, so that NHS ester that had not undergone a reaction was blocked. The solution was centrifuged at 8000×g and 4° C. for 30 minutes. The supernatant was removed so that approximately 1 mL of the solution remained and then the gold colloid was dispersed again using an ultrasonic washing machine. Thereafter, the resultant was dispersed in 20 mL of a gold colloidal stock solution (20 mM Tris-HCl buffer (pH 8.2), 0.05% PEG (Mw. 20000), 150 mM NaCl, 1% BSA, and 0.1% NaN₃) and then centrifuged again at 8000×g and 4° C. for 30 minutes. The supernatant was removed so that approximately 1 mL of the solution remained and then the gold colloid was dispersed again using an ultrasonic washing machine. Thus, an antibody-labeled gold colloidal solution was obtained.

By confirming that no antibody was eluted when the gold colloid was mixed with a 0.1 % SDS solution, it was confirmed that the antibody could be immobilized via the SH group to the thus prepared labeled gold colloid using PEG polymer.

### (3) Preparation of Gold Colloidal Antibody Holding Pad

The antibody-labeled gold colloids prepared in Comparative example 1 and Examples 1 and 2 were each diluted with a coating solution for a gold colloid (20 mM Tris-Hcl buffer (pH 8.2), 0.05 % PEG (Mw. 20000), and 5 % sucrose) and water to set the OD at 520 nm to 3.0. The thus diluted anti-virus A antibody-labeled gold colloidal solution and the anti-virus B antibody-labeled gold colloidal solution were mixed at a ratio of 1:1. The solution was uniformly applied to glass fiber pads cut to the size of 8 mm×150 mm in an amount of 0.8 mL per pad. The pads were dried under reduced pressure overnight to obtain gold colloidal antibody holding pads.

### (4) Preparation of Antibody-Immobilized Membrane (Chromatographic Carrier)

Antibody-immobilized membranes prepared under completely the same conditions were used in the present invention. An antibody-immobilized membrane was prepared in the following manner by immobilizing an antibody on a nitrocellulose membrane (HiFlow Plus HF 120 with a plastic lining, Millipore Corporation) cut to the size of 25 mm×200 mm. A membrane, with one of its long sides facing downward, was coated with an anti-influenza A virus antibody solution prepared to a concentration of 1.5 mg/ml with the use of a coater of inkjet type, so that a linear portion thereof (7 mm above the lower edge) having a width of approximately 1 mm was coated. In a similar manner, a membrane was coated with an anti-influenza B virus antibody solution prepared to a concentration of 1.5 mg/ml with the use of a coater of inkjet type, so that a linear portion thereof 10 mm above the lower edge was coated to have a width of approximately 1 mm. Furthermore, a membrane was coated with a control anti-mouse IgG antibody solution prepared to a concentration of 0.5 mg/mL, so that a linear portion thereof 13 mm above the lower edge was coated. Each coated membrane was dried at 50° C. for 30 minutes with a hot-air dryer. The membrane was immersed in 500 ml of a blocking solution (50 mM borate buffer (pH 8.5) containing 0.5 w % casein (milk-derived product, Product No. 030-01505, Wako Pure Chemical Industries, Ltd.)) in a vat and then allowed to stand therein for 30 minutes. Thereafter, the membrane was transferred to and immersed in 500 ml of a washing-stabilizing solution (0.5 w % sucrose, 0.05 w % sodium cholate, and 50 mM Tris-HCl (pH 7.5)) in a similar vat and then allowed to stand therein for 30 minutes. The membrane was removed from the solution and then dried overnight at room temperature to prepare an antibody-immobilized membrane.

### (5) Assembly of Kit

The thus prepared antibody-immobilized membrane was adhered to a back pressure-sensitive adhesive sheet (ARcare9020, NIPPN TechnoCluster, Inc.). At this time, the membrane was used with the anti-influenza A virus antibody line side (one of the long sides of the membrane) facing downward. The prepared gold colloidal antibody holding pad was adhered onto the antibody-immobilized membrane such that the pad overlapped the lower portion of the antibody-immobilized membrane by approximately 2 mm. A sample addition pad (glass fiber pad (Glass Fiber Conjugate Pad, Millipore Corporation) cut to the size of 18 mm×150 mm) was adhered to the gold colloidal antibody holding pad such that the sample addition pad overlapped the lower portion of the gold colloidal antibody holding pad by approximately 4 mm. Furthermore, an absorbent pad (cellulose membrane cut to the size of 5 mm×100 mm (Cellulose Fiber Sample Pad, Millipore Corporation)) was adhered onto the antibody-immobilized membrane such that the absorbent pad overlapped the upper portion of the antibody-immobilized membrane by approximately 5 mm. With the use of a guillotine cutter (CM4000, NIPPN TechnoCluster, Inc.), the thus overlapped and adhered members were cut in parallel to the short sides of the overlapped members at 5-mm intervals, whereby strips for immunochromatography having a width of 5 mm were prepared. These strips were placed in a plastic case (NIPPN TechnoCluster, Inc.), so as to prepare an immunochromatographic kit for testing.

### (6) Measurement Method

### 1. Preparation of Silver Amplification Solution

First, 40 mL of 1 M iron nitrate aqueous solution (prepared by dissolving iron (III) nitrate nonahydrate (Product No. 095-00995, Wako Pure Chemical Industries, Ltd.) in 325 g of water), 10.5 g of citric acid (Product No. 038-06925, Wako Pure Chemical Industries, Ltd.), 0.1 g of dodecylamine (Product No. 123-00246, Wako Pure Chemical Industries, Ltd.), and 0.44 g of surfactant C₉H₁₉—C₆H₄—O—(CH₂CH₂O)₅₀H were mixed for dissolution. After they had all dissolved, 40 mL of nitric acid (10% by weight) was added to the solution while stirring it using a stirrer. 80 mL of the solution was weighed and then 11.76 g of iron (II) ammonium sulfate hexahydrate (Product No. 091-00855, Wako Pure Chemical Industries, Ltd.) was added to the solution. The thus obtained solution was designated solution I.

Next, water was added to 10 mL of a silver nitrate solution (containing 10 g of silver nitrate) to a total amount of 100 g. The thus obtained solution was designated solution II.

Finally, 4.25 mL of solution II was added to 40 mL of solution I, followed by stirring, thereby preparing a silver amplification solution.

### 2. Method for Measuring the Amount of Antigen Bound

The following experiment was conducted for all the kits prepared in Comparative example 1 and Examples 1 and 2.

As antigens, Quick S-Influ A•B “Seiken” negative/positive controls (Product No. 322968, DENKA SEIKEN Co., Ltd.) were used. First, the positive control was diluted to 1/640 with PBS buffer containing 1% BSA. Next, 100 μL of the diluted antigen solution was applied dropwise to a prepared kit and then the kit was allowed to stand for 10 minutes. Subsequently, the membrane was removed from the case and then placed in a microtube (Product No. BM4020, BM Equipment Co., Ltd.) containing 700 μL of a washing solution so that the portions at which the sample had been applied dropwise were immersed in the solution, so as to wash the membrane for 1 hour.

A water absorbent pad was removed and then three fresh water absorbent pads (5 mm×20 mm) (Cellulose Fiber Sample Pad, Millipore Corporation)) were caused to adhere to the portion (from which the pad had been removed) using cellophane tape. The membrane was placed in a microtube containing 200 μL of an amplification solution, so that the portions to which the sample had been applied dropwise were immersed in the solution. The time point at which the membrane had absorbed the amplification solution so that the amplification solution reached the detection line was determined to be 0 minutes. Two minutes after the time point (0 minutes), each membrane was removed. The amounts of gold adsorbed to antibody-coated portions (detection line) of the membranes were measured based on the thus detected shading (dark or light) of the black precipitates. The degrees of color development at detection lines of the membranes after silver amplification were quantified using an immunochromato-reader ICA-1000 (Hamamatsu Photonics K.K.). Table 1 shows the results.

In this experiment, the fluid level (fluid height) that depends on the tube shape upon washing, the shape and material of a sample addition pad in the immunochromatographic kit, the experimental environment (temperature and humidity), the material and thickness of an absorbent pad, the joint between an absorbent pad and a nitrocellulose membrane, and the like are factors that alter the water absorption speed and amount of a lavage fluid. Hence, it is required in this experiment to keep them at constant levels. The water absorption speed and amount of the washing solution are factors that affect the final effects of washing (reduction of the amount of the remaining fine gold particles). The experiment of the present invention was conducted at a temperature of 24±3° C. and humidity of 45±8%.

### 3. Measurement of the Amount of Nonspecific Adsorption

The following experiment was conducted for all the kits prepared in Comparative example 1 and Examples 1 and 2.

100 μL of PBS buffer containing 1% BSA was spotted instead of the positive control used in 2. Method for measuring the amount of antigen bound. The experiment was conducted in the same manner as in 2. above except the use of PBS buffer. Table 1 shows the results.

As a result of comparing the results each obtained by dividing the amount of antigen bound (A) by the level of nonspecific adsorption (B) ((A)/(B)) as in Table 1, the labeled particle of the present invention was confirmed to be extremely effective.

TABLE 1

| | Absorbance (mABS) | | |
|---|---|---|---|
| | Comparative | | |
| | Example 1 | Example 1 | Example 2 |
| Amount of antigen bound | 19.6 | 24.4 | 30.2 |
| (A) | | | |
| Amount of nonspecific | 0.8 | 0.4 | 0.1 |
| adsorption (B) | | | |
| (A)/(B) | 24.5 | 61.0 | 302.0 |

1. A labeled particle, wherein a fragmented antibody is immobilized to a labeling substance via a chemical bond.

2. The labeled particle according to claim 1, wherein the fragmented antibody is an Fab fragment and/or an Fab′ fragment and/or an F (ab′)₂ fragment.

3. The labeled particle according to claim 2, wherein the fragmented antibody is directly bound to the labeled particle, or is bound to the labeled particle via a hydrophilic polymer.

4. The labeled particle according to claim 3, wherein the hydrophilic polymer contains an ethylene glycol group in at least a portion thereof.

5. The labeled particle according to claim 4, wherein the polymer containing an ethylene glycol group in at least a portion thereof is at least one type selected from among polyethylene glycol and derivatives thereof.

6. The labeled particle according to claim 1, wherein the fragmented antibody is bound to the labeled particle via an SH group of an antibody.

7. The labeled particle according to claim 1, wherein the labeling substance is a metal colloid.

8. The labeled particle according to claim 7, wherein the metal colloid is a gold colloid, a silver colloid, or a platinum colloid.

9. A sandwich immunochromatographic method which comprises developing a complex formed of an analyte and a labeled particle for the analyte on a porous carrier and capturing the analyte and the labeled particle at a reaction site on the porous carrier that has a second antibody against the analyte so as to detect the analyte, wherein the labeled particle is the labeled particle of claim 1.

10. The immunochromatographic method according to claim 9, wherein a labeling substance having an average particle size of 1 μm or more and 20 μm or less is detected.

11. The immunochromatographic method according to claim 9, wherein an analyte is detected via sensitization using a silver-containing compound and a reducing agent for silver ions.

12. The immunochromatographic method according to claim 9, wherein the reaction time for sensitization using the silver-containing compound and the reducing agent for silver ions is within 7 minutes.

13. The immunochromatographic method according to claim 9, wherein the number of the labeling substance at a detection site is 1×10⁶/mm³ or less.

14. The immunochromatographic method according to claim 9, wherein the labeling substance is a metal colloid.
